(19) United States
(12) Patent Application Publication
(10) Pub. No.: US 2010/0034885 A1
(43) Pub. Date: Feb. 11, 2010

(54) FORMULATIONS CONTAINING GLIMEPIRIDE AND/OR ITS SALTS

(75) Inventor: Marta Tarruella Delsams, Lleida (ES)

Correspondence Address: IP Patent Docketing;K&L GATES LLP, 599 Lexington Avenue, 33rd Floor, New York, NY, 10022-6030, US

(73) Assignee: COMBINO PHARM, S.L., Barcelona (ES)

(21) Appl. No.: 11/921,887

(22) PCT Filed: May 30, 2006
(86) PCT No.: PCT/IB2006/003998

§ 371 Date: Oct. 23, 2009

Related U.S. Application Data
(60) Provisional application No. 60/689,091, filed on Jun. 10, 2005.

(51) Int. Cl. A61K 9/14; A61K 31/40; A61P 3/10

(52) U.S. Cl. 424/489; 514/424

(57) ABSTRACT

The invention relates, in general, to new formulations and dosage units containing glimepiride of defined particle size and/or salts thereof that are useful for the therapeutic treatment (including prophylactic treatment) of mammals, including humans, without the need for micronizing any excipients together with the glimepiride that advantageously saves time, energy and resources and a process for making the same. In particular, the invention can be useful for the treatment of diabetes.

### CROSS REFERENCE TO RELATED APPLICATIONS

This application claims priority to U.S. Provisional Application No. 60/689,091, filed Jun. 10, 2005, which application is expressly incorporated herein by reference in its entirety.

### BACKGROUND OF THE INVENTION

1. Field of the Invention

The invention relates, in general, to new formulations and dosage units containing glimepiride of defined particle size and/or salts thereof that are useful for the therapeutic treatment (including prophylactic treatment) of mammals, including humans, without the need for micronizing any excipients together with the glimepiride that advantageously saves time, energy and resources and a process for making the same. In particular, the invention can be useful for the treatment of diabetes.

2. Relevant Background

Diabetes is characterized by excessive urine excretion. Type II diabetes (i.e., non-insulin dependent diabetes mellitus; “NIDDM”) is the most common type of diabetes. This form of diabetes is caused by either (a) an insufficient production of insulin in the pancreas (relative insulin deficiency), (b) a resistance to the action of insulin in the body's cells (insulin resistance), especially in muscle, fat and liver cells, or (c) an increased hepatic production of glucose.

Uncontrolled Type II diabetes results in excess glucose accumulation in the blood which causes hyperglycemia (i.e., high blood sugar). In some cases, Type II diabetes can be managed by creating a balance between a healthy diet, regular physical activity and maintaining a healthy body weight. Over time, however, the condition may require oral medications.

Several classes of oral antidiabetic agents have been shown to lower blood glucose levels. Such antidiabetic agents include sulfonylureas, which increase insulin secretion and potentiate insulin action on the liver and peripheral tissues; metformin, which decreases hepatic glucose production, increases glucose uptake and possibly decreases appetite; alpha glucosidase inhibitors, which slow the absorption of carbohydrates; troglitazone, which decreases insulin resistance; and others.

Glimepiride (chemical name: N-[4-[2-(3-ethyl-4-methyl-2-oxo-3-pyrroline-1-carboxamido)-ethyl]-benzenesulfonyl]-N′-4-methylcyclohexylurea or 1-[[p-[2-(3-ethyl-4-methyl-2-oxo-3-pyrroline-1-carboxamido)ethyl]phenyl]sulfonyl]-3-(trans-4-methylcyclohexyl)urea) is an antidiabetic medication of the sulfonylureas class that is used to treat Type II diabetes. Glimepiride lowers blood sugar levels by stimulating the production and release of insulin from the pancreas. It also promotes the movement of sugar from the blood into the cells in the body that need it. Glimepiride has the following formula:

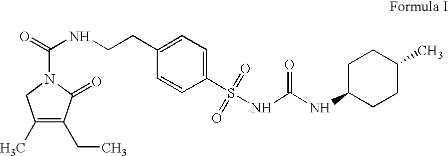

Glimepiride is polymorphic, and two forms, Form I and Form II, have been isolated and characterized to date as reported in Acta Cryst., C53, 329-331 (1997) and S.T.P. Pharma Sciences, 13 (4) 281-286 (2003), respectively, which are each incorporated herein by reference.

Glimepiride is currently marketed under the name AMARYL® and is indicated as an adjunct to diet and exercise for lowering blood glucose levels in patients having non-insulin dependent diabetes mellitus (NIDDM) or Type II diabetes and whose hyperglycemia cannot be controlled by diet and exercise alone.

U.S. Pat. No. 4,379,785 (“the '785 patent”) discloses heterocyclic substituted sulfonylureas, including glimepiride. The '785 patent further indicates that glimepiride has hypoglycemic properties and is suitable for use as medicaments (e.g., as an antidiabetic agent). The '785 patent also indicates that formulations containing glimepiride and/or salts thereof can be administered orally for the treatment of diabetes mellitus and that suitable medicament formulations are preferably tablets containing the usual carriers and excipients such as talc, starch, lactose or magnesium stearate. The '785 patent also discloses that it may be advantageous to use the active substance(s) in ground or finely dispersed form, or as a mixture of these two forms, although it does not provide any details regarding such ground substances to be used in formulations having good bioavailability.

EP 0 649 660 relates to pharmaceutical preparation for enteral administration of virtually water-insoluble medicinal substances, including glimepiride, and a process for its production. In particular, the medicinal preparation described contains a medicinal substance that is virtually insoluble in water and/or lipophilic media and one or more physiologically tolerated amphosurfactant(s) that is/are water-soluble or soluble in water in a micellar-colloidal manner, which substances are present in dissolved form in one or more physiologically tolerated, water-free and water-miscible solvent(s). Thus, this patent addresses the problem of delivery of low solubility compounds/formulations by using particular excipients that increase solubility.

Changes in particle size can affect the solubility properties for compounds exhibiting poor aqueous solubility (e.g., glimepiride) and/or poor bioavailability. In particular, a reduction in particle size may improve a compound's solubility as a result of increasing the ratio of the solid's surface area that is in contact with the aqueous liquid medium. Notably, however, particle size reduction cannot alter the solubility of a compound in a solvent, which is thermodynamically controlled.

It is known in the art that in some instances the rate of dissolution of a poorly soluble drug is the rate limiting factor in its rate of absorption by the body. It is also recognized that such drugs may be more readily bioavailable if administered in a finely divided state.

Particle size can also affect how freely the crystals or a powdered form of a drug will flow past each other when processed and thus is of consequence in the production processes of pharmaceutical products containing the same.

In pharmaceutical products, the particle size of drugs and excipients affect processing and bioavailability. Particle size reduction resulting in an increased surface area, is a very promising approach to enhance dissolution rate and, consequently, the bioavailability of poorly water soluble drugs, such as glimepiride. One of the problems associated with the milling of a compound is the formation of agglomerates. One approach to addressing this problem is to include excipients when milling the active ingredient. This approach is used, for example, in WO 2004/082591 which describes milling the active ingredient or a mixture of the active ingredient with one or more excipients in order to obtain a pharmaceutical formulation that is bioequivalent with a commercially available pharmaceutical formulation of glimepiride. In the examples present in WO 2004/082591, glimepiride is milled together with some excipients until the milled material passes through a 60 mesh ASTM sieve (250 μm).

Additionally, it is desirable to have a uniform distribution of the active ingredient within a dosage unit. Traditionally, active ingredients are randomly distributed within a dosage unit. Recently, however, efforts have been directed to blending processes for specifically arranging particles within a blend. In this regard, it has been observed that the randomness and the arrangement of particles can yield blends of different characteristics. In practice, this may be important because in the pharmaceutical industry, blending may be carried out in small fractions that constitute the dosage form (e.g., tablets, capsules). Likewise, the blending sequence of the components can affect both the uniformity as well as others properties such as, for example, the mechanical strength and/or the biodisponibility.

It is an object of the invention to provide new formulations and dosage units containing glimepiride of defined particle size and/or salts thereof that are useful for the therapeutic treatment (including prophylactic treatment) of mammals, including humans, and a process for making the same. In particular, the invention can be useful for the treatment of diabetes.

### SUMMARY OF THE INVENTION

The invention provides new formulations and dosage units containing glimepiride of defined particle size and/or salts thereof that are useful for the therapeutic treatment (including prophylactic treatment) of mammals, including humans, without the need for micronizing any excipients together with the glimepiride that advantageously saves time, energy and resources and a process for making the same. In particular, the invention can be useful for the treatment of diabetes.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition, and as will be appreciated by one of skill in the art, the invention may also be embodied as a method, system or process.

The invention provides new formulations and dosage units containing glimepiride of defined particle size and/or salts thereof that are useful for the therapeutic treatment (including prophylactic treatment) of mammals, including humans, and a process for making the same. These formulations and processes avoid the need for micronizing any excipients together with the glimepiride, which advantageously saves time, energy and resources.

The formulations and/or dosage units of the invention include a therapeutically acceptable quantity of glimepiride and/or salts thereof (e.g., 1, 2, 3, 4 or 6 mg) and further include one or more pharmaceutically acceptable carriers and/or excipients.

In particular, the invention provides a pharmaceutical composition comprising micronized glimepiride particles having a median particle size (D₅₀) by volume equal to or less than approximately 3.00 μm (measured by light scattering) and a pharmaceutically acceptable carrier.

As discussed above, using these smaller particle sizes helps improves the homogeneity of the pharmaceutical formulation. Traditionally, such methodologies would employ a co-micronizing method, which involves co-milling the active ingredient with one or more excipients. The invention does not require co-micronizing and eliminates the need to micronize the other components of the formulation. In other words, the active ingredient glimepiride is the only component of the composition that requires micronization. Consequently, the invention minimizes or eliminates agglomerate formation that can negatively affect the bioavailability of the pharmaceutical formulation. Surprisingly, micronized glimepiride having the above-described particle sizes is easily manageable and can be formulated into dosage units using conventional equipment and thus avoids the need to use extreme measures or specialized technology to achieve and maintain relatively tiny particles to facilitate dissolution and bioavailability and promote homogeneity of the formulations.

In one aspect of the invention, the glimepiride particles in the composition have a D₉₀ not exceeding approximately 10.00 μm. As used herein, the notation Dₓ means that X % by volume of the particles have a diameter less than a specified diameter. Thus, for example, a D₉₀ of approximately 10.00 μm means that approximately 90% of the particles by volume in a composition preferably have a diameter less than approximately 10.00 μm.

In another aspect of the invention, the glimepiride particles have a median particle size (D₅₀) by volume that is equal to or less than approximately 3.00 μm, more preferably equal to or less than approximately 2.50 μm, even more preferably equal to or less than approximately 2.00 μm, and most preferably equal to or less than approximately 1.50 μm according to Coulter light scattering.

In another aspect of the invention, the glimepiride particles in the composition have a D₉₀ not exceeding approximately 10.00 μm, more preferably not exceeding approximately 5.00 μm, even more preferably not exceeding approximately 3.60 μm according to Coulter light scattering.

Another aspect of the invention includes a process for preparing pharmaceutical formulations that includes the steps of (i) micronizing glimepiride to obtain a median particle size (D₅₀) equal to or less than about 3 μm and (ii) combining the micronized glimepiride with at least one suitable excipient by a wet granulation process.

In another aspect of the invention, the above compositions are used in a method for treating a Type II diabetes mellitus that includes administering to a patient in need of thereof an effective amount of a composition which includes micronized glimepiride having a median particle size (D₅₀) equal to or less than approximately 3.00 μm as measured by Coulter light scattering and a pharmaceutically acceptable carrier.

Particle sizes can be determined by laser light scattering techniques using a Coulter Model LS 130 particle size analyzer (with a Microvolume unit attached) (discussed below).

The pharmaceutical compositions of the invention advantageously exhibit good dissolution properties at physiologic pH. In particular, the pharmaceutical formulations of the invention that include glimepiride particles having a median particle size (D₅₀) equal to or less than approximately 3.00 μm exhibit bioequivalency with currently available commercial pharmaceutical compositions of glimepiride. Thus, according to one aspect of the invention, glimepiride can easily be formulated with glimepiride particles having a median particle size (D₅₀) equal to or less than approximately 3.00 μm and that can be used with conventional formulation equipment and methodologies without the need to use extreme measures and/or specialized technology to achieve and/or maintain relatively tiny particles to facilitate dissolution.

In another aspect of the invention, the pharmaceutical formulations according to this invention, when tested in vitro, exhibit improved dissolution characteristics. Specifically, glimepiride formulations according to the invention exhibit the following dissolution properties: 70% of glimepiride (in a formulation containing 6 mg or less of glimepiride) dissolves within 15 minutes in a 900 mL solution of 0.05 M NaH₂PO₄ buffer, adjusted to approximately pH 6.6 (e.g., by the addition of diluted NaOH or diluted phosphoric acid), containing 0.2% (w/w) sodium dodecyl sulfate and which is placed in a USP-2 apparatus equipped with paddles stirring at 50 rpm. This testing protocol is established as an average for a pre-determined number (e.g., six) of dosages (i.e., tablets), and the dissolution media is typically maintained at approximately 37° C. during the test. The amount of dissolved glimepiride can be determined conventionally by HPLC, as hereinafter described.

The bioequivalent pharmaceutical compositions according to the invention minimally include glimepiride having a median particle size (D₅₀) equal to or less than approximately 3.00 μm and, although such formulations may also include one or more pharmaceutically acceptable excipient(s), such excipient(s) are not required to be micronized with the glimepiride. Thus, according to another aspect of the invention, the process of micronizing the glimepiride can be optimized (i.e., because the glimepiride can be micronized alone), and the homogeneity of the average glimepiride particle size can be more easily controlled.

As used herein, the term “particles” refers to individual particles regardless of whether the particle(s) exist singly or are agglomerated. Thus, a composition comprising particulate glimepiride may contain agglomerates that are well beyond the size limit of about 3.00 μm specified herein. If, however, the median size of the primary drug substance particles comprising the agglomerate is less than approximately 3.00 μm individually, then the agglomerate itself is considered to satisfy the particle size constraints defined herein.

As used herein, the term “pharmaceutical composition” means a medicament for use in treating a mammal formulated in tablet form and which includes micronized glimepiride having a median particle size (D₅₀) equal to or less than approximately 3.00 μm and at least one pharmaceutically acceptable excipient.

Bioavailability is the rate and extent to which the active substance (i.e., glimepiride) is absorbed from a pharmaceutical formulation and becomes available in general circulation. Bioavailability is assessed by serial measurements of the drug in systemic circulation. These serial measurements provide a plasma concentration/time curve from which important pharmacokinetic parameters can be calculated, including, for example, the area under the curve (AUC), the maximum observed concentration (Cₘₐₓ) and the time when Cₘₐₓ is reached (Tₘₐₓ). The AUC provides an estimate of the amount of drug absorbed in the systemic circulation, while Tₘₐₓ and Cₘₐₓ reflect the rate of absorption.

As used herein, two medicinal products are considered to be bioequivalent when their bioavailabilities after administration in the same dose under similar conditions in a comparative, randomized, open-label, single-dose, 2-way crossover study are similar. The degree of similarity between two formulations is determined by the appropriate statistical assessment and by meeting the following criteria: the 90% confidence interval of the relative mean AUC of the test to reference product should be within 80 to 125%. The same criteria should be met for Cₘₐₓ: the 90% confidence interval of the relative mean measured Cₘₐₓ of the test to reference should be within 80 to 125%.

Glimepiride suitable for use in the invention can be obtained by any reasonable synthetic route, including those routes described in EP 0 031 058, which is incorporated by reference herein. Additionally, any of the polymorphic forms of glimepiride (i.e., Form I or Form II) may be used in the formulations of invention. In the discussion and illustrative examples that follow, glimepiride Form I was used and is referred to throughout as glimepiride unless noted otherwise.

Glimepiride of defined particle size can, for example, be produced by precipitation from appropriate solvents. Under such conditions, precipitation rates and particle size can be controlled by customary methods including, for example, cooling, pH adjustment, pouring a concentrated solution of glimepiride into an anti-solvent and/or by co-precipitation in order to obtain glimepiride with an appropriate average surface area by volume.

Glimepiride of defined particle size can also be produced by other known techniques and methodologies (described below) for reducing the particle size of crystals, powder aggregates and/or coarse powders. Such methodologies include, for example, milling of a feedstock material and sorting of milled materials by size (e.g., sieving).

A fluid energy mill, or “micronizer” is an especially preferred type of mill for preparing particles of small size and having a narrow size distribution. Micronizers use the kinetic energy of collision between particles suspended in a rapidly moving fluid (e.g., air) stream to cleave the particles.

An air jet mill is one preferred fluid energy mill in which suspended particles are injected under pressure into a recirculating particle stream. The smaller particles are carried aloft inside the mill and swept into a vent connected to a particle size classifier (e.g., cyclone). Prior to using an air jet mill, the feedstock material is generally first milled to approximately 150 to 850 μm using conventional methods (e.g., a conventional ball, roller, or hammer mill).

Another method for preparing particles of small size and having a narrow size distribution is sorting milled materials by passing the same through a stack of sieves, each with openings of a different and diminishing size.

Glimepiride particles of well-defined size can also be separated by particle size using cyclonic or centrifugation techniques.

Average particle size was measured using a Coulter Model LS 130 laser light scattering analyzer (with a Microvolume unit attached) and a laser beam of 4 mW and 750 nm wavelength. Samples of the glimepiride were suspended in water containing a surfactant (e.g., 0.125% Tween 80). The suspensions were mixed together and sonicated for approximately 300 seconds to thoroughly disperse the glimepiride particles, and the sample cell was equipped with a magnetic agitation system to ensure that the sample remains suspended during testing.

Samples for analysis were prepared by adding a weighed amount of glimepiride (approximately 10±0.1 mg) and approximately 10 mL of a previously prepared suspending media (which includes an aqueous solution of 0.125% (by volume) of Tween 80) in a 50 mL glass vial. The glimepiride was suspended in this solution by sonicating in an ultrasonic bath for approximately 5 minutes. Prior to sample analysis, a background count was achieved by filling the measurement cell with 15 mL of the suspending media without any glimepiride present. For sample analysis, a disposable Pasteur pipette was used to first withdraw and empty portions of the suspension several times to ensure representative sampling of the sample vial contents. The pipette was next filled and a few drops of the vial contents were added to the suspending medium in the measurement cell until an obscuration value of approximately 12% was obtained. Next, the intensity of the light scattered by the suspended sample was measured at different angles by an array of detectors. According to the Fraunhoffer model of light scattering by particles, a volume distribution of the suspended sample was obtained. The calculations were performed by the software accompanying the Coulter LS 130 apparatus.

Suitable excipients for use in the invention can include conventional pharmaceutically acceptable excipients including, for example, fillers and diluents (e.g., starches and sugars), binders (e.g., carboxymethyl cellulose and other cellulose derivatives, alginates, gelatin, and polyvinyl pyrrolidone), disintegrating agents (e.g., agar-agar, calcium carbonate, sodium bicarbonate, pregelatinized starch, corn starch, algenic acid, sodium croscarmellose, sodium starch glycolate and crosslinked polyvinylpyrrolidone), lubricants (e.g., talc, sodium lauryl sulfate, stearic acid, calcium and magnesium stearate, and solid polyethyl glycols). Some excipients can serve more than one function; for example, a disintegrant can also function as a filler.

For tablet formulations, it is typically preferable to include one or more benign pharmaceutical excipients in the composition. In this regard, powder compositions of the invention can include one or more diluents to make the tablet larger and, hence, easier for the patient and/or caregiver to handle. Suitable diluents for use in the invention include, for example, microcrystalline cellulose, microfine cellulose, lactose, starch, pregelatinized starch, calcium carbonate, calcium sulfate, sugar, dextrates, dextrin, dextrose, dibasic calcium phosphate dihydrate, tribasic calcium phosphate, magnesium carbonate, sodium carbonate, maltodextrin, mannitol, potassium chloride, powdered cellulose, sodium chloride, sorbitol and talc.

Binders can be included to facilitate tablet stability after compression. Suitable binders for use in the invention include, for example, acacia, algenic acid, carbomer, carboxymethylcellulose sodium, cellulose microcrystalline, dextrin, ethyl cellulose, gelatin, guar gum, hydrogenated 4 vegetable oil, hydroxyethyl cellulose, hydroxypropyl cellulose, hydroxypropyl methyl cellulose, liquid glucose, magnesium aluminum silicate, maltodextrin, methylcellulose, polymethacrylates, povidone, pregelatinized starch, sodium alginate and starch.

The formulations of the invention can further include a disintegrant to help accelerate disintegration of the tablet in the patient's stomach. Suitable disintegrants for use in the invention include, for example, algenic acid, carboxymethyl cellulose calcium, carboxymethylcellulose sodium, colloidal silicon dioxide, croscarmellose sodium, crospovidone, guar gum, methyl cellulose, microcrystalline cellulose, polacrilin potassium, powdered cellulose, pregelatinized starch, sodium alginate, sodium starch glycolate and starch.

The formulations of the invention can further include glidants, lubricants, flavorings, colorants, preservatives and other commonly used excipients. Suitable lubricating agents for use in the invention include, for example, magnesium stearate, stearic acid and/or talc. Suitable preservative agents for use in the invention include, for example, ethyl or propyl p-hydroxy benzoate. Suitable anti-oxidants for use in the invention include, for example, ascorbic acid.

Alternative equivalent excipients (i.e., other release control agents, fillers, lubricants, binders, etc.) having the same and/or similar functions and/or properties may be readily substituted and used in the below illustrative formulations.

The formulations of the invention can be prepared as tablets by using conventional methodologies and employing conventional equipment for preparing the same.

In one aspect of the invention, a preferred manufacturing process includes (i) sizing and removing “lumps” from each of micronized glimepiride, lactose monohydrate, a first portion (approximately 70-75%) of sodium starch glycolate and, optionally, a first portion (approximately 85-90%) of a dye by either (a) sieving through a medium mesh size or (b) gently milling using common stainless steel sieves or mechanical mills; (ii) mixing of the sized components in a suitable blender (e.g., a drum, container, high performance, planetary, bicone or V-blender or granulator) to ensure good homogeneity; (iii) adding a prepared solution of povidone in water under mixing to the powder blend obtained in step (ii) using either a vertical or horizontal high shear granulator or low speed granulator until a suitable consistency is achieved; (iv) drying the wet mass (e.g., by using a fluid bed drier, oven tray, vacuum or vacuum-microwave driers); (v) calibrating the dried materials using a medium mesh sieve in a common stainless steel siever or a mechanical mill; (vi) adding and blending microcrystalline cellulose, the balance of sodium starch glycolate and the balance of dye; (vii) adding and blending magnesium stearate (though blending should be continued for no more than 20 minutes); (viii) optionally sampling the mixture; (ix) preparing tablet formulations by compression (e.g., using a rotary or eccentric press) while making any dose necessary adjustments to tablet weight; and (x) optionally coating the tablets with a suitable coating material.

In the above-described process, the dry blend can be performed in a suitable mixer, such as a container blender, drum blender, v-blender or a high shear mixer. Tablet compression can be performed in a tablet press, and the optional coating process can be performed in a coating pan or fluid bed.

The initial therapy dosage of glimepiride is 1 mg once daily, administered with breakfast or the first main meal. Usual maintenance dosages are between 1 and 4 mg once daily. The maximum recommended dose is 6 mg once daily. Thus, in another aspect of the invention, the amount of glimepiride contained in each tablet of the invention is between approximately 1 and approximately 6 mg for use once daily. In another aspect of the invention, the invention includes tablets having amounts of glimepiride outside this range and/or at different frequencies of administration.

A tablet can be tested to assess its dissolution profile and characteristics by methodology described above.

The amount of dissolved glimepiride can be determined conventionally by HPLC using a suitable chromatographic column (e.g., a Symmetry C-18 5 μm 4.6×250 mm column) with an isocratic mobile phase consisting of 1300 mL of acetonitrile and 700 mL of potassium dihydrogen phosphate buffer, pH 3.0 and a flow rate of approximately 1.0 mL/min at room temperature. Detection can be accomplished using UV absorption at 228 nm. Data is quantified by comparison of the HPLC peak area relative to the peak area taken from a standard plot of concentration versus peak area for standards of known concentration. In this regard, glimepiride standard concentrations are selected to fall within a linear range of concentration versus absorbance for the UV detector employed.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

The following examples are for illustrative purposes only and are not intended, nor should they be interpreted to, limit the scope of the invention.

### EXAMPLE 1

### Formulation of Glimepiride Tablet (2 mg)

Table 1 (below) illustrates a representative tablet formulation containing 2 mg of glimepiride according to one aspect of the invention.

TABLE 1
Composition of Tablet Containing 2 mg of Glimepiride

| | kg | mg |
|---|---|---|
| Component | (Per 100,000 tablets) | (Per tablet) |
| Glimepiride | 200 | 2.000 |
| Lactose Monohydrate | 14.690 | 146.920 |
| Sodium Starch Glycolate | 1.360 | 13.600 |
| Green Dye 1035 | 18 | 0.180 |
| Povidone | 90 | 0.900 |
| Microcrystalline Cellulose | 53 | 0.530 |
| Sodium Starch Glycolate | 500 | 5.000 |
| Green Dye 1035 | 2 | 0.020 |
| Magnesium Stearate | 85 | 0.850 |

| | |
|---|---|
| TOTAL | 170 |

All the components were sieved through 0.8 mm mesh except the dye, which was sieved through 0.25 mm mesh. Glimepiride, lactose, approximately one half of the total amount of the dye and 500 g of sodium starch glycolate were mixed and blended using a drum blender for approximately 30 minutes. The mixture was placed into a high shear granulator. Following blending, a solution of povidone at 10% (w/w) in water was added to the granulator and blending continued for 10 minutes and until the mixture achieved adequate homogeneity. The wet mass was then calibrated through a 2 mm mesh sieve and then dried in a fluid bed at approximately 40° C. The resulting dry granulate was calibrated through a 0.8 mm mesh sieve. Separately, the balance of (or, as needed, additional) dye and the balance of (or, as needed, additional) sodium starch glycolate were combined and the mixture was blended in a drum blender for approximately 30 minutes. Following blending, this mixture was added to the calibrated granulate and blending was continued for approximately 15 additional minutes. Following blending, magnesium stearate was added and blending continued for an additional 5 minutes. The final blend was then compressed into tablets using a rotary press.

### EXAMPLES 2-4

### Formulations of Glimepiride Tablet (3, 4 and 6 mg)

Glimepiride tablets having 3, 4 and 6 mg, respectively, of active pharmaceutical ingredient were formulated as described in Example 1 to achieve a total tablet weight of approximately 170 mg. The increased quantity of glimepiride was offset by a decrease in the quantity of lactose monohydrate (i.e., 3 mg of glimepiride: 145.920 mg lactose; 4 mg glimepiride: 144.920 mg lactose; 6 mg glimepiride: 143.12 mg lactose (no dye is used in 6 mg formulation so the balance includes additional lactose)).

### EXAMPLE 5

### Formulation of Glimepiride Tablet (1 mg)

Glimepiride tablets having 1 mg of active pharmaceutical ingredient were formulated as described in Example 1 by using half the amount of each excipient to achieve a total tablet weight of approximately 85 mg.

### EXAMPLE 6

### Dissolution of Glimepiride Tablet (2 mg)

The tablets of Example 1 and commercially available glimepiride tablets (i.e., AMARYL® 2 mg) were tested for in vitro drug release in 900 mL of 0.05 M NaH₂PO₄ buffer, having a pH of approximately 6.6 and containing 0.2% (w/w) sodium dodecyl sulphate. A USP-2 apparatus with paddle speed at 50 rpm was used for the study. The dissolution results are reported in Table 2 (below) and illustrated in Graph 1 (below):

TABLE 2

| Time | Tablet Example 1 | AMARYL ® |
|---|---|---|
| (minutes) | % Drug Release Profile | % Drug Release Profile |
| 5 | 81.11 | 78.74 |
| 10 | 97.69 | 91.23 |
| 15 | 102.58 | 95.09 |
| 20 | 103.29 | 95.79 |
| 30 | 104.61 | 97.85 |
| 45 | 105.02 | 98.51 |
| 60 | 104.18 | 99.15 |

### EXAMPLE 7

### Bioavailability of Glimepiride Tablet (2 mg)

The bioavailability of glimepiride tablets (2 mg) prepared according to the invention was evaluated in a single center, single dose, open-label, randomized, two way crossover, bioequivalence study under fasting conditions. The bioavailability study compared the glimepiride tablets (2 mg) with commercially marketed glimepiride (i.e., Amaryl® 2 mg) administered as single 2 mg dosages in order to evaluate the comparative rates and extent of absorption thereof.

The bioavailability study included a total of 40 healthy volunteers male and females, between 18 and 55 years of age. Plasma samples from the first 38 subjects completing the study were analyzed and used for pharmacokinetic and statistical analysis. Both tablets were administered as single doses, with a washout period of 14 days, and samples were taken to determine the glimepiride plasma levels. Blood samples were collected at hour 0 (pre-dose) and at 0.5, 1, 1.5, 2, 2.25, 2.5, 2.75, 3, 3.5, 4, 5, 6, 8, 10, 12, 16, 24, and 36 hours post-dose. In all, 19 samples were taken per subject and treatment and the glimepiride plasma levels were analyzed using a validated LC/MS/MS method.

The main evaluation variables for the bioavailability assessment were: AUC₀₋ₜ, AUC_{0-inf}, Cₘₐₓ, Tₘₐₓ. Individual analysis of variance (ANOVA) were performed on the ln-transformed data of AUC₀₋ₜ, AUC_{0-inf} and Cₘₐₓ. All ANOVAs were performed with the SAS General Liner Models Procedure (GLM). Tables 3 and 4 report the results of the bioavailability study.

TABLE 3

| | Glimepiride Tablet (2 mg) | | | Amaryl ® (2 mg) | | |
|---|---|---|---|---|---|---|
| | (Example 1) | | | (Reference) | | |
| Parameters | Mean | SD | CV (%) | Mean | SD | CV (%) |
| AUC₀₋ₜ | 581.67 | 222.72 | 38.29 | 563.31 | 219.09 | 38.89 |
| (ng · h/mL) | | | | | | |
| AUC_{0-inf} | 608.49 | 242.47 | 39.85 | 586.51 | 234.78 | 40.03 |
| (ng · h/mL) | | | | | | |
| Cₘₐₓ (ng/ml) | 127.87 | 42.20 | 33.00 | 130.35 | 37.66 | 28.89 |
| Residual | 3.96 | 3.36 | 84.84 | 3.67 | 2.35 | 64.07 |
| area (%) | | | | | | |
| Tₘₐₓ (h) | 2.48 | 0.55 | 22.18 | 2.42 | 0.55 | 22.56 |
| Tₘₐₓ* (h) | 2.50 | 0.69 | — | 2.50 | 0.69 | — |

*For Tₘₐₓ medians and interquartile ranges are also presented.

TABLE 4

| | AUC₀₋ₜ | AUC_{0-inf} | Cₘₐₓ |
|---|---|---|---|
| Ratio LS | 102.98% | 103.31% | 97.39% |
| Means | | | |
| 90% Geometric | 98.85%-107.29% | 99.22%-107.57% | 89.05%-106.51% |
| C.I. | | | |

According to the study protocol (described above) and the Guidance on Bioavailability and Bioequivalence (“Note for guidance on the investigation of bioavailability and bioequivalence” (CPMP/EWP/QWP/1401/98)), bioequivalence of a formulation is established if the 90% geometric confidence intervals of the least-square means ratios of the test to reference products of ln-transformed AUC₀₋ₜ and Cₘₐₓ were within an acceptance range of 80% to 125%. Table 4 demonstrates that the glimepiride tablets 2 mg prepared according to the invention meets this criteria relative to the reference glimepiride tablets (i.e., Amaryl® 2 mg). Namely, this study demonstrates that the ratio for AUC₀₋ₜ, the test versus reference is 98.85% to 107.29%; for AUC_{0-inf}, test versus reference is 99.22% to 107.57%; and for Cₘₐₓ, the test versus reference is 89.05% to 106.51%. Thus, according to this data, the glimepiride tablet (2 mg) obtained as described in Example 1 is bioequivalent to the reference glimepiride tablet (i.e., Amaryl® 2 mg).

What is claimed is:

1. A pharmaceutical formulation comprising:
(i) a therapeutically effective amount of glimepiride and/or its pharmaceutically acceptable salts; and
(ii) at least one pharmaceutically acceptable excipient,
wherein said glimepiride and/or its pharmaceutically acceptable salts is micronized and wherein said glimepiride and/or its pharmaceutically acceptable salts has a median particle size (D₅₀) equal to or less than approximately 3 μm.

2. The pharmaceutical formulation to claim 1, wherein said median particle size (D₅₀) of said glimepiride and/or its pharmaceutically acceptable salts is between approximately 2.5 μm and approximately 1 μm.

3. The pharmaceutical formulation of claim 2, wherein said median particle size (D₅₀) of said glimepiride and/or its pharmaceutically acceptable salts is between approximately 1.5 μm and approximately 1 μm.

4. The pharmaceutical formulation according to claim 1, wherein the maximum particle size of said glimepiride and/or its pharmaceutically acceptable salts is less than approximately 20 μm; and
wherein approximately 90% by volume of the particles of said glimepiride and/or its pharmaceutically acceptable salts have a diameter less than approximately 10.0 μm.

5. The pharmaceutical formulation of claim 4, wherein approximately 90% by volume of the particles of said glimepiride and/or its pharmaceutically acceptable salts have a diameter less than approximately 5 μm.

6. The pharmaceutical formulation of claim 5, wherein approximately 90% by volume of the particles of said glimepiride and/or its pharmaceutically acceptable salts have a diameter less than approximately 3.6 μm.

7. The pharmaceutical formulation according to claim 1, wherein said glimepiride and/or its pharmaceutically acceptable salts constitutes between approximately 1% to approximately 4% by weight of said pharmaceutical formulation.

8. The pharmaceutical formulation of claim 1, wherein said at least one pharmaceutically acceptable excipient comprises at least one of a binder material, a filler material, a disintegrant material, a lubricant material, a glidant material, a granulating agent, a release control agent, a preservative agent, an anti-oxidant agent and combinations thereof.

9. The pharmaceutical formulation of claim 1, wherein said pharmaceutical formulation comprises between approximately 1 mg and approximately 6 mg of said glimepiride and/or its pharmaceutically acceptable salts.

10. The pharmaceutical formulation according to claim 1, wherein said at least one pharmaceutically acceptable excipient is at least one of starches, sugars, carboxymethyl cellulose and other cellulose derivatives, alginates, gelatin, polyvinyl pyrrolidone, agar-agar, calcium carbonate, sodium bicarbonate, pregelatinized starch, corn starch, algenic acid, sodium croscarmellose, sodium starch glycolate, crosslinked polyvinylpyrrolidone, talc, sodium lauryl sulfate, stearic acid, calcium stearate, magnesium stearate, solid polyethyl glycols, microcrystalline cellulose, microfine cellulose, lactose, starch, calcium sulfate, sugar, dextrates, dextrin, dextrose, dibasic calcium phosphate dihydrate, tribasic calcium phosphate, magnesium carbonate, sodium carbonate, mannitol, potassium chloride, powdered cellulose, sodium chloride, sorbitol, acacia, carbomer, carboxymethylcellulose sodium, ethyl cellulose, guar gum, hydrogenated 4 vegetable oil, hydroxyethyl cellulose, hydroxypropyl cellulose, hydroxypropyl methyl cellulose, liquid glucose, magnesium aluminum silicate, maltodextrin, methylcellulose, polymethacrylates, povidone, sodium alginate, carboxymethyl cellulose calcium, colloidal silicon dioxide, croscarmellose sodium, crospovidone, guar gum, methyl cellulose, polacrilin potassium, powdered cellulose, sodium alginate, sodium starch glycolate, stearic acid, ethyl p-hydroxy benzoate, propyl p-hydroxy benzoate and ascorbic acid.

11. The pharmaceutical formulation according to claim 1 formulated as a tablet.

12. A process for preparing a pharmaceutical formulation comprising a therapeutically effective amount of glimepiride and/or its pharmaceutically acceptable salts comprising:
(i) micronizing said glimepiride and/or its pharmaceutically acceptable salts until the median particle size (D₅₀) of said glimepiride and/or its pharmaceutically acceptable salts is equal to or less than about 3 μm; and
(ii) combining said micronized glimepiride and/or its pharmaceutically acceptable salts with at least one pharmaceutically acceptable excipient;
wherein said micronized glimepiride and/or its pharmaceutically acceptable salts and said at least one pharmaceutically acceptable excipient are combined by a wet granulation process.

13. The process of claim 12, wherein said at least one pharmaceutically acceptable excipient comprises at least one of a binder material, a filler material, a disintegrant material, a lubricant material, a glidant material, a granulating agent, a release control agent, a preservative agent, an anti-oxidant agent and combinations thereof.

14. The process of claim 12, wherein said at least one pharmaceutically acceptable excipient is at least one of starches, sugars, carboxymethyl cellulose and other cellulose derivatives, alginates, gelatin, polyvinyl pyrrolidone, agar-agar, calcium carbonate, sodium bicarbonate, pregelatinized starch, corn starch, algenic acid, sodium croscarmellose, sodium starch glycolate, crosslinked polyvinylpyrrolidone, talc, sodium lauryl sulfate, stearic acid, calcium stearate, magnesium stearate, solid polyethyl glycols, microcrystalline cellulose, microfine cellulose, lactose, starch, calcium sulfate, sugar, dextrates, dextrin, dextrose, dibasic calcium phosphate dihydrate, tribasic calcium phosphate, magnesium carbonate, sodium carbonate, mannitol, potassium chloride, powdered cellulose, sodium chloride, sorbitol, acacia, carbomer, carboxymethylcellulose sodium, ethyl cellulose, guar gum, hydrogenated 4 vegetable oil, hydroxyethyl cellulose, hydroxypropyl cellulose, hydroxypropyl methyl cellulose, liquid glucose, magnesium aluminum silicate, maltodextrin, methylcellulose, polymethacrylates, povidone, sodium alginate, carboxymethyl cellulose calcium, colloidal silicon dioxide, croscarmellose sodium, crospovidone, guar gum, methyl cellulose, polacrilin potassium, powdered cellulose, sodium alginate, sodium starch glycolate, stearic acid, ethyl p-hydroxy benzoate, propyl p-hydroxy benzoate and ascorbic acid.

15. The process according to claim 12, wherein said wet granulation process comprises:
(i) blending said micronized glimepiride and/or its pharmaceutically acceptable salts with a first quantity of at least one filler, a first quantity of a disintegrant, and optionally at least one other excipients to form a mixture;
(ii) granulating the mixture of step (i) with an aqueous solution of at least one binder;
(iii) drying and sieving the granulated mixture of step (ii);
(iv) blending the mixture of step (iii) with a second quantity of at least one filler and the remaining quantify of disintegrant, and optionally other excipients;
(v) blending the mixture of step (iv) with at least one lubricant; and
(vi) compressing the drug mixture of step (v) into a pharmaceutical dosage form.

16. The process of claim 15, wherein said first quantity of at least one filler, said first quantity of a disintegrant, said at least one other excipient, said at least one binder, said second quantity of at least one filler and said at least one lubricant are each at least one of starches, sugars, carboxymethyl cellulose and other cellulose derivatives, alginates, gelatin, polyvinyl pyrrolidone, agar-agar, calcium carbonate, sodium bicarbonate, pregelatinized starch, corn starch, algenic acid, sodium croscarmellose, sodium starch glycolate, crosslinked polyvinylpyrrolidone, talc, sodium lauryl sulfate, stearic acid, calcium stearate, magnesium stearate, solid polyethyl glycols, microcrystalline cellulose, microfine cellulose, lactose, starch, calcium sulfate, sugar, dextrates, dextrin, dextrose, dibasic calcium phosphate dihydrate, tribasic calcium phosphate, magnesium carbonate, sodium carbonate, mannitol, potassium chloride, powdered cellulose, sodium chloride, sorbitol, acacia, carbomer, carboxymethylcellulose sodium, ethyl cellulose, guar gum, hydrogenated 4 vegetable oil, hydroxyethyl cellulose, hydroxypropyl cellulose, hydroxypropyl methyl cellulose, liquid glucose, magnesium aluminum silicate, maltodextrin, methylcellulose, polymethacrylates, povidone, sodium alginate, carboxymethyl cellulose calcium, colloidal silicon dioxide, croscarmellose sodium, crospovidone, guar gum, methyl cellulose, polacrilin potassium, powdered cellulose, sodium alginate, sodium starch glycolate, stearic acid, ethyl p-hydroxy benzoate, propyl p-hydroxy benzoate and ascorbic acid.

17. The process of claim 15, wherein said at least one filler of step (i) is lactose monohydrate.

18. The process of claim 15, wherein said at least one binder of step (ii) is povidone.

19. The process of claim 15, wherein said at least one filler of step (iv) is microcrystalline cellulose.

20. The process of claim 15, wherein said at least one lubricant of step (v) is magnesium stearate.

21. The process of claim 15, wherein said at least one disintegrant is an intragranular disintegrant.

22. The process of claim 15, wherein said at least one disintegrant is an extragranular disintegrant.

23. The process of claim 15, wherein said at least one disintegrant is sodium starch glycolate.

24. A method of improving the bioavailability of a pharmaceutical formulation comprising glimepiride and/or its pharmaceutically acceptable salts, the method comprising:
(i) micronizing said glimepiride and/or its pharmaceutically acceptable salts until its median particle size (D₅₀) is equal to or less than about 3 μm;
(ii) combining said micronized glimepiride and/or its pharmaceutically acceptable salts with at least one pharmaceutically acceptable excipient; and
(iii) compressing said micronized glimepiride and/or its pharmaceutically acceptable salts and said at least one pharmaceutically acceptable excipient into a pharmaceutical dosage formulation,
wherein said micronized glimepiride and/or its pharmaceutically acceptable salts is the only micronized component of said pharmaceutical dosage formulation.
